(19) United States
(12) Patent Application Publication
(10) Pub. No.: US 2019/0184235 A1
(43) Pub. Date: Jun. 20, 2019

(54) METHOD AND PROGRAM FOR DETERMINING TRAINING RATIO

(71) Applicant: NEOFECT CO., LTD., Yongin-si, Gyeonggi-do (KR)

(72) Inventors: Hyo Seok YI, Yongin-si, Gyeonggi-do (KR); Hyun Soo KIM, Seoul (KR); Ho Yeong SONG, Yongin-si, Gyeonggi-do (KR)

(21) Appl. No.: 16/327,440

(22) PCT Filed: Aug. 16, 2017
(86) PCT No.: PCT/KR2017/008872

§ 371 Date: Feb. 22, 2019

(30) Foreign Application Priority Data
Aug. 24, 2016 (KR) 10-2016-0107986

(51) Int. Cl. A63B 24/00; G16H 20/30

(52) U.S. Cl. CPC A63B 24/0075; A63B 2024/0012; G16H 20/30; A63B 24/0006

(57) ABSTRACT

The present disclosure relates to a method and a program for determining a training ratio. A method for determining a training ratio according to an embodiment of the present disclosure includes acquiring current state data for a specific body part of a specific user S100; acquiring state evaluation data by applying the current state data and normal state data to a current state evaluation model S200; calculating training level data for a specific training type by applying the acquired state evaluation data to a training ratio determination model S300; and determining a performance ratio by calculating a ratio of training level data for multiple training types S400. According to the present disclosure, a user can be provided with a rehabilitation training curriculum optimized for his/her rehabilitation at other locations than a medical institution.

### TECHNICAL FIELD

The present disclosure relates to a method and a program for determining a training ratio of multiple training types when rehabilitation training is performed.

### BACKGROUND

Currently, there are many patients in need of function recovery training due to their spinal cord injury caused by car accident, brain injury caused by cerebral infarction or stroke, or dysfunction of a specific body part caused by other causes. In general, patients with dysfunction of a specific body part perform various gradual function recovery trainings with the help of a rehabilitation therapist or a nurse.

However, when the patients perform the function recovery trainings, i.e., rehabilitation trainings, with the help of a rehabilitation therapist or a nurse, they have to visit the hospital to perform the rehabilitation trainings. Further, since the patients perform a simple rehabilitation training with the help of a rehabilitation therapist or a nurse, they become bored with the rehabilitation training.

Accordingly, rehabilitation trainings based on game content have been introduced. However, even if a patient performs rehabilitation training based on game content, a nurse or a rehabilitation therapist needs to select game content suitable for the patient. Therefore, the patient needs to visit the hospital to perform an optimum rehabilitation training based on the selected game content.

### DISCLOSURE OF THE INVENTION

### Problems to be Solved by the Invention

The present disclosure provides a method and a program for determining a training ratio by which a training performance ratio suitable for a patient among multiple training types for a body part in need of rehabilitation training is determined and provided, and, thus, the patient can perform an optimum rehabilitation training without visiting a medical institution to receive help from a medical staff.

However, problems to be solved by the present disclosure are not limited to the above-described problems. Although not described herein, other problems to be solved by the present disclosure can be clearly understood by a person with ordinary skill in the art from the following description.

### Means for Solving the Problems

According to an aspect of the present disclosure, a method for determining a training ratio includes: acquiring current state data for a specific body part of a specific user; acquiring state evaluation data by applying the current state data and normal state data to a current state evaluation model; calculating training level data for a specific training type by applying the acquired state evaluation data to a training ratio determination model; and determining a performance ratio by calculating a ratio of training level data for multiple training types, and the current state data is data for performance of the specific training type for the specific body part of the user, the normal state data is data for performance of the specific training type for the specific body part of a normal person, and the state evaluation data is data that is evaluated by comparing the current state data with the normal state data to determine a training level suitable for the current state of the user.

Further, the current state evaluation model may calculate the state evaluation data by calculating a third value corresponding to the state of the user according to a specific calculation equation in a numerical range between a first numerical value corresponding to a minimum state and a second numerical value corresponding to a normal person, and the calculation equation may correspond to features of the specific body part.

Furthermore, the training ratio determination model may set the training level data to 0 in the minimum state and a normal state and have an equation which has a specific training level data value in a specific state between the minimum state and the normal state and is set differently depending on the body part or the training type.

Moreover, if the body part is a hand, the training type may include rotating a wrist, bending and stretching a wrist, rotating a forearm, and folding and unfolding a finger, and the current state data may be measurement data of a range of motion of a joint for the training type and the determining of the performance ratio may include calculating the performance ratio of the multiple training types.

Further, if the specific training type has a motion that is symmetrical to a reference posture, the state evaluation data of the specific training type may be calculated by averaging first state evaluation data corresponding to a motion in a first direction and second state evaluation data corresponding to a motion in a second direction.

Furthermore, the normal state data may be a maximum point on a graph of the number of normal persons for performance result data of the specific training type or an average value of the performance result data acquired from multiple normal persons.

Moreover, the square of a real number greater than 1 is applied to a difference value between the normal state data and the current state data.

Besides, the determining of the performance ratio may include: acquiring a reference ratio of the multiple training types; and calculating the performance ratio by multiplying a value corresponding to the specific training type within the reference ratio and the training level data of the specific training type.

Further, if there are multiple determination criteria for calculating the performance ratio for each training type, the performance ratio for each of the multiple determination criteria may be calculated.

Furthermore, the method may further include: generating a composite ratio for the multiple training types by multiplying multiple values corresponding to the respective training types within the performance ratio for each of the multiple determination criteria.

According to another aspect of the present disclosure, a program for determining a training ratio is combined with hardware to perform the above-described method for determining a training ratio and stored in a medium.

### Effects of the Invention

According to the present disclosure, users can be provided with a rehabilitation training curriculum suitable for their state through a computer without visiting a medical institution to perform rehabilitation training. That is, the computer can evaluate the current state of a user for each training type and adjust a performance ratio of each training type, and, thus, the user can be provided with a rehabilitation training curriculum optimized for his/her rehabilitation at other locations than a medical institution.

Further, since a current state evaluation model and a training ratio determination model optimized for each body part and each training type are used, it is possible to calculate a performance ratio of multiple training types applied with features of each body part and training type can be calculated.

### BRIEF DESCRIPTION OF THE DRAWINGS

FIG. 1 is a flowchart showing a method for determining a training ratio according to an embodiment of the present disclosure.

FIG. 2 is an example graph showing the distribution for normal persons to calculate normal state data according to an embodiment of the present disclosure.

FIG. 3 is an example graph by a calculation equation in a current state evaluation model according to an embodiment of the present disclosure.

FIG. 4 is an example graph by a training ratio calculation model according to an embodiment of the present disclosure.

FIG. 5 is a flowchart showing a process of calculating a performance ratio by applying training level data to a reference ratio according to an embodiment of the present disclosure.

FIG. 6 is a flowchart showing a method for determining a training ratio that further includes a process of calculating a composite ratio combining performance ratios for respective multiple determination criteria according to an embodiment of the present disclosure.

### BEST MODE FOR CARRYING OUT THE INVENTION

Hereafter, desirable embodiments of the present disclosure will be described in detail with reference to the accompanying drawings. The advantages and characteristics of the present disclosure and a method of achieving the advantages and characteristics will be clear by referring to exemplary embodiments described below in detail together with the accompanying drawings. However, the present disclosure is not limited to exemplary embodiment disclosed herein but will be implemented in various forms. The exemplary embodiments are provided by way of example only so that a person with ordinary skill in the art can fully understand the disclosures of the present disclosure and the scope of the present disclosure. Therefore, the present disclosure will be defined only by the scope of the appended claims. Throughout the whole specification, the same reference numerals denote the same elements.

Unless otherwise defined, all terms (including technical and scientific terms) used herein have the same meaning as commonly understood by a person with ordinary skill in the art to which this invention pertains. It will be further understood that terms, such as those defined in commonly used dictionaries, should not be interpreted in an idealized or overly formal sense unless expressly so defined herein.

The terms used herein provided only for illustration of the exemplary embodiments but not intended to limit the present disclosure. As used herein, the singular terms include the plural reference unless the context clearly indicates otherwise. The terms “comprises” and/or “comprising” specify the presence of stated components, but do not preclude the presence or addition of one or more other components., and the term “and/or” includes any and all combinations of one or more of the associated listed items.

In the present specification, a “computer” includes all various devices that can provide a user with a result by performing a calculation. For example, the computer may correspond to a smartphone, a tablet Pc, a cellular phone, a personal communication service (PCS) phone, a mobile terminal of a synchronous/asynchronous international mobile telecommunication (IMT)-2000, a palm personal computer (PC), and a personal digital assistant (PDA) as well as a desktop PC and a notebook. Further, the computer may be a server that receives a request from the client and processes information.

In the present specification, “training” is an act that is performed to improve or enhance a function of a specific body part of a user. That is, when a function of a body part of a specific user does not reach the state of a normal person (i.e., when the user is a patient in need of rehabilitation for a specific body part), the “training” is rehabilitation training that is performed to improve the specific body part. Further, the specific user corresponds to a normal person, and when the user wants to further improve the function of the body part, function improving exercise (for example, muscular exercise, muscle endurance exercise, and the like) performed on the specific body part corresponds to the “training”.

In the present specification, a “training type” is a type that has to be performed as training of a specific body part. The “training type” may mean a type of a motion that may be performed by a specific body part. For example, if the body part is a “hand”, the “training type” includes folding and unfolding a finger, bending and stretching a wrist, rotating a wrist, and the like. Further, the “training type” may mean a detailed type of a task (for example, orange squeezing game content and butterfly catching game content for “folding and unfolding a finger”) that moves a specific body part.

If training is performed at a hospital, rehabilitation therapists at the hospital help patients with training. In this case, a rehabilitation therapist determines a training type or a training difficulty level suitable for a patient and also determines the sequence of trainings to be performed and the frequency of training. Therefore, if training is performed at a medical care center such as a hospital, a rehabilitation training system including multiple training types does not need to set a type of training to be performed, the sequence of performing trainings, and the like.

However, if a user (i.e., a patient) personally performs training using the rehabilitation training system without the help of a rehabilitation therapist, he/she cannot determine a training type, the sequence of performing trainings, and the frequency of each training required for himself/herself. Accordingly, a system, a method, and a program that enable a user to personally perform a suitable training at home without the help of a rehabilitation therapist are needed.

Hereafter, a system, a method, and a program for determining a training ratio according to embodiments of the present disclosure will be described in detail with reference to the accompanying drawings.

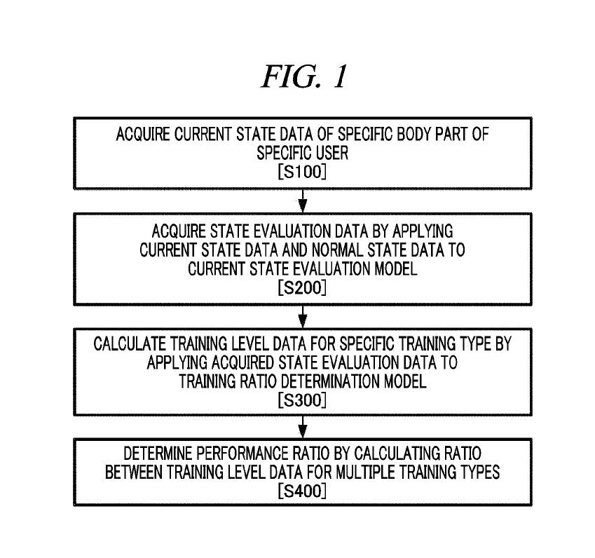

FIG. 1 is a flowchart showing a method for determining a training ratio according to an embodiment of the present disclosure.

Referring to FIG. 1, the method for determining a training ratio according to an embodiment of the present disclosure includes: acquiring current state data for a specific body part of a specific user (S100); acquiring state evaluation data by applying the current state data and normal state data to a current state evaluation model (S200); calculating training level data for a specific training type by applying the acquired state evaluation data to a training ratio determination model (S300); and determining a performance ratio by calculating a ratio of training level data for multiple training types (S400). Hereafter, each of the processes will be described in detail.

A computer acquires current state data for a specific body part of a specific user (S100). The current state data is data for performance of a specific training type for the specific body part of the user. That is, the computer acquires data for determining the current state of a body part of the user (e.g., a patient).

In an embodiment, when a range of motion (ROM) of a joint is measured, current state data is acquired by measuring a current specific motion of the joint of the user. The range of motion of the joint may be measured using a rehabilitation training device (for example, a body state measuring device or a glove type/hand-worn rehabilitation device) that performs training while being worn on a specific body part of the user. For example, if the body part is a hand, the training type includes rotating a wrist, bending and stretching a wrist, rotating a forearm, folding and unfolding a finger, and the computer acquires measurement data of a range of motion of a joint for the training type as current state data when the user performs a motion while wearing a hand-worn measurement device. Further, for example, when a range of motion of a shoulder joint is measured, the range of motion of the joint is measured using a measurement sensor device attached to the shoulder part or a device placed on the bottom surface to provide a 2-dimensional motion of the shoulder.

In another embodiment, a medical staff or a rehabilitation therapist measures a modified ashworth scale (MAS) from the patient and performs a manual muscle testing (MMT) to the patient to measure the muscular stiffness of the patient and his/her exercise ability in the direction of gravity, and the computer receives the measurement data and calculates specific numerical data corresponding to current state data.

The computer acquires state evaluation data by applying the current state data and normal state data to a current state evaluation model (S200). First, the computer acquires the normal state data for the specific training type for the specific body part. The normal state data is data for performance of the specific training type for the specific body part of a normal person.

The computer may acquire normal state data in various schemes. As an embodiment, a manager designates normal state data of a specific training type to the computer. Specifically, when the computer is a terminal device of the user, it receives and stores optimum normal state data as it performs wireless communication with an external management server.

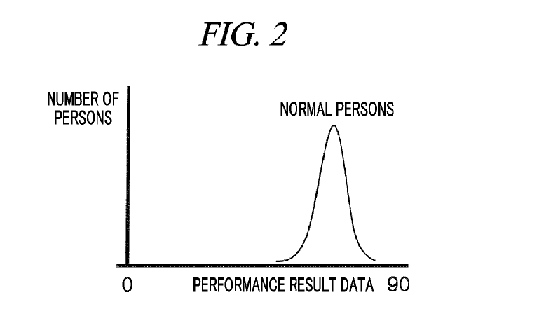

Further, in another embodiment, the normal state data is determined to be a maximum point on a graph of the number of normal persons for the performance result data of a specific training type. Specifically, if the computer is a management server, it may accumulate performance results for a specific training type of normal persons as shown in FIG. 2 and generate a graph of the number of normal persons for the corresponding performance result data. The performance result data value on the graph, which corresponds to the largest number of persons may be determined as normal state data. Further, in another embodiment, the normal state data is determined to be an average value of the performance result data acquired by multiple normal persons.

The state evaluation data is data that is evaluated by comparing the current state of the user with the state of a normal person to determine a training level suitable for the current state of the user. It may not be proper to directly apply, to calculation of a training ratio, current state data measured by a body state measurement device or a rehabilitation device or current state data that is a result of a specific test performed to the user by a medical staff. To this end, the computer converts the current state data into data suitable for calculating a training ratio. That is, the computer performs a process of evaluating the current state, by comparing the current state with the state of a normal person.

In an embodiment of the current state evaluation model, the state evaluation data is calculated by calculating a third numerical value corresponding to the state of the user according to a specific calculation equation in a numerical range between a first numerical value corresponding to a minimum state and a second numerical value corresponding to a normal person. That is, the calculation equation may be an equation for an increasing function or a decreasing function that changes between the first numerical value corresponding to the minimum state (i.e., the state that does not perform a training type at all) and the second numerical value corresponding to the normal state, and the computer may apply, to the calculation equation, the third numerical value corresponding to specific current state data between the minimum state and the normal state.

The calculation equation included in the current state evaluation model may be a functional equation that increases or decreases in direct proportion, and may be an equation that is in conformity with a specific function other than a linear function. For example, in a motion of rotating a wrist, a motion of a small rotation angle may be determined as a high improvement in state within an initial rotation zone in a specific direction, but in a zone close to a maximum rotation range, an improvement of the performance result by the same rotation angle may be determined as a low improvement in state as compared with the initial rotation zone. Accordingly, when the state of a patient is evaluated, the same difference value of the current state data measured previously and the current state data measured currently may be determined differently depending on the previous state (i.e., the rate of change in the state evaluation data may be different).

For example, an example of calculating state evaluation data based on the following equation will be described in detail. Meanwhile, the equation of the current state evaluation model according to the present disclosure is not limited thereto.

$${z}_{i} = \frac{1}{2 {\overline{n}}^{1.2}} {( {x}_{i} - \overline{n} )}^{1.2}$$

n̅: normal state data, xᵢ: current state data, and zᵢ: state evaluation data

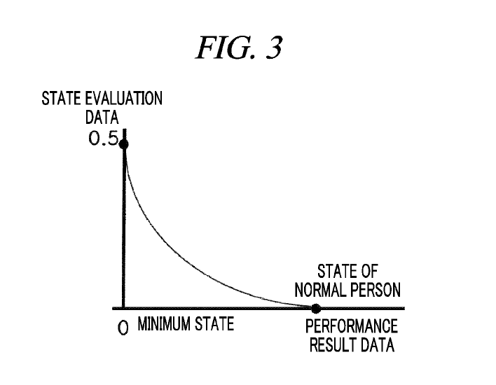

The numerical range of the equation is set from 0 to 0.5 and becomes closer to a maximum value of 0.5 as the state becomes worse (i.e., the state becomes closer to a minimum state) and becomes closer to 0 as the state becomes better (i.e., xᵢ becomes closer to n). Further, since the equation has a form of an exponential function or a functional form (i.e., a graph form before a minimum point that is downwardly convex as shown in FIG. 3) in which functional values decrease while the rate of change increases as xᵢ (i.e., current state data) increases, a difference of the state evaluation data values according to the same difference value of the current state data in an area in which the state becomes better (i.e., an area that is close to that of a normal person) is smaller than a difference of state evaluation data according to a difference value (e.g., an increase of a motion range of rotation of a wrist by a specific angle) of specific current state data in an area in which the state is not good (i.e., an area that is close to that of the minimum state).

An embodiment of the calculation equation in a current state evaluation model in which a functional value decreases while the rate of change increases as current state data increase may be an equation that applies the square of a real number greater than 1 to a difference value between the normal state data and the current state data.

Further, as an embodiment, the computer applies an equation corresponding to features of a specific body part or a training type as the calculation equation included in the current state evaluation model. Different equation forms may be applied while reflecting the features according to the body part or the training type, or only a constant value included in the same equation form may be adjusted.

Further, in another embodiment, when a specific training type has a motion that is symmetrical to a reference posture, the state evaluation data of the specific training type is calculated by averaging first state evaluation data corresponding to a motion in a first direction and second state evaluation data corresponding to a motion in a second direction. For example, if the training type is rotating a wrist, since a wrist can be rotated in opposite directions from a reference state (i.e., the state in which a joint is not rotated), the current states for the respective directions (i.e., first direction and second direction) have to be evaluated individually. Further, for example, if the training type is bending a wrist, the bending of the wrist in upward and downward directions has to be evaluated individually. That is, the computer acquires state evaluation data (i.e., first state evaluation data and second state evaluation data) for the respective directions (i.e., first direction and second direction) of the body part having a symmetric motion. Thereafter, the computer acquires final state evaluation data of a specific training type of the corresponding body part by averaging the first state evaluation data and the second state evaluation data.

Further, in another embodiment, if the training type has a symmetrical motion of a specific body part, the current state evaluation model includes an equation of calculating, as state evaluation data, a numerical value that reflects the states for the respective directions. For example, If the training type is rotating a wrist, there are a first state in which the wrist is inclined by 30 degrees in the first direction from a reference location and is not rotated at all in the second direction and a second state in which the wrist is inclined by 15 degrees in the first direction and inclined by 15 degrees in the second direction, the final state evaluation data of the first state and the final state evaluation data of the second state may be calculated in the same way by applying an equation of a function that is in direct proportion to a deviation between the normal state data and the current state data (i.e., an equation in which the state evaluation data increase in direct proportion to the normal state data). When the state of the patient is evaluated during rehabilitation training, the first state and the second state need to be evaluated as being different and to be displayed as the numerical values of the final state evaluation data, and, thus, the computer uses an equation in which a deviation from the calculated final state evaluation data values in a specific direction is reflected.

In an embodiment of the current state evaluation model for this, an equation for applying the square of a real number greater than 1 to a difference value between the normal state data and the current state data is included. For example, as shown in FIG. 3, in an equation having a functional form in which a functional value decreases while the rate of change increases as current state data increase, since an absolute value of the rate of change for the state evaluation data increases as the current state data decrease (i.e., the state evaluation data rapidly change as the current state data is small), the deviation of the motion in a specific direction can be applied to the numerical values.

The computer calculates training level data for a specific training type by applying the acquired state evaluation data to a training ratio determination model (S300). The training level data means a training level of the user that is suitable for state evaluation data of a specific training type of a specific body part. The training ratio of multiple training types needs to be differently set according to the state of a patient. For example, the training type in which the patient produces a performance result close to a normal person may be provided less and the training type in which the patient may be determined to be improved with a high possibility may be provided with an increased training ratio to increase the training effect.

In an embodiment of the training ratio determination model, training level data in the minimum state and the normal state is set to 0 and the training ratio determination model has an equation with a specific training level data value in a specific state between the minimum state and the normal state. When a specific training type is performed, in the minimum state (for example, the state in which a specific body part cannot move at all according to a specific training type), the corresponding training type cannot be helpful to the user and may deteriorate the user's interest in the rehabilitation training. Accordingly, the computer sets the training level data in the minimum state to 0. Further, when state evaluation data of a training type for a specific body part is calculated to be equivalent to a level of a normal person, the corresponding user does not require rehabilitation of the training type for the corresponding body part. Therefore, the computer sets the training level data of the corresponding training type to 0. Further, an equation is determined to have a specific continuous function between the minimum state and the normal state. An embodiment of the continuous function may be a function of determining training level data based on a state evaluation data value in a specific numerical range (i.e., a range of greater than 0 and smaller than 1). The equation included in the training ratio calculation model may be set differently depending on the body part or the training type.

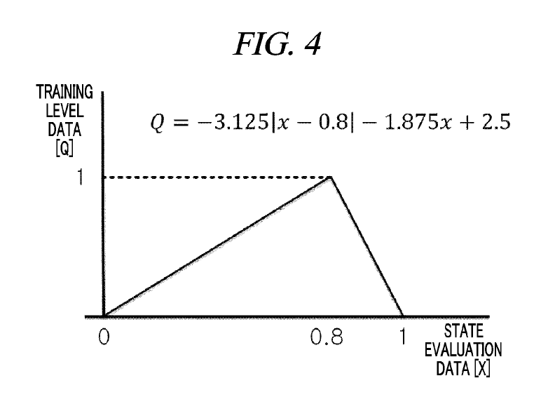

For example, as shown in FIG. 4, the equation may be included in the training ratio determination model such that a maximum training ratio (i.e., maximum value) is obtained at a specific state evaluation data value. The state evaluation data value having a maximum value may be set by a medical staff or may be set and adjusted by analyzing rehabilitation levels (i.e., degree of improvement in the state) of the users by the computer.

The computer determines a performance ratio of multiple training types based on training level data for the multiple training types (S400). In an embodiment of a method for calculating the performance ratio, the computer calculates a ratio by directly comparing training level data of the respective training types. For example, if the training level data of training type A, training type B, and training type C are 0.6, 0.5, and 0.4, respectively, the computer determines 0.6:0.5:0.4 that is a ratio between the training level data for the respective training types as a performance ratio. Further, the computer may calculate a final ratio in the form of a ratio of integers by multiplying the performance ratio by a specific natural number.

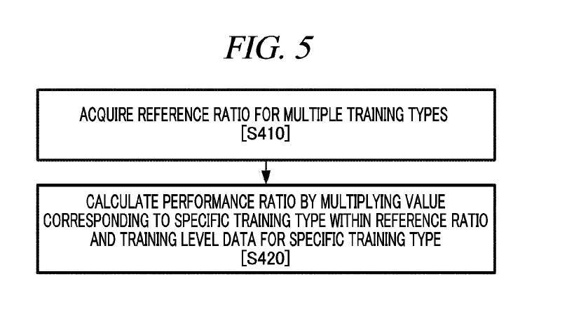

Further, in another embodiment, the computer includes a basic training ratio, and calculates a performance ratio corresponding to the current state of the user by multiplying a value corresponding to each training type within the basic training ratio and training level data of each training type. As an embodiment of the method for calculating the performance ratio based on the basic training ratio, the determining of the performance ratio (S400) may include: acquiring a reference ratio of the multiple training types (S410); and calculating a performance ratio by multiplying a value corresponding to a specific training type within the reference ratio and training level data of the specific training type, as shown in FIG. 5.

The computer acquires a reference ratio of multiple training types (S410). For example, when a body part to which the user has to perform rehabilitation training is determined, the computer extracts multiple training types to be performed to the body part and extracts reference ratios which are basically set to be achieved for the multiple training types.

Then, the computer calculates a performance ratio by multiplying a value corresponding to a specific training type within the reference ratio and training level data of the specific training type (S420). For example, if the training level data of training type A, training type B, and training type C are 0.6, 0.5, and 0.4, respectively, and a reference ratio applied to training type A, training type B, and training type C is 3:2:1, a performance ratio is calculated by multiplying a ratio corresponding to each training type and training level data (i.e., by performing (3*0.6):(2*0.5):(1*0.4)).

Further, if there are multiple determination criteria for calculating a performance ratio for each training type, a performance ratio for each of the multiple determination criteria may be calculated. The multiple determination criteria include a range of motion (ROM) of a join, muscular stiffness (the level at which the muscle can move by itself), exercise ability in the direction of gravity, cognitive ability, and the like. In an embodiment, if the training type is an exercise performance form (e.g., rotating a wrist, bending a wrist, or the like), performance ratios for multiple exercise performance forms are calculated according to each of the determination criteria. That is, different performance ratios for the same exercise performance form may be calculated according to the determination criteria.

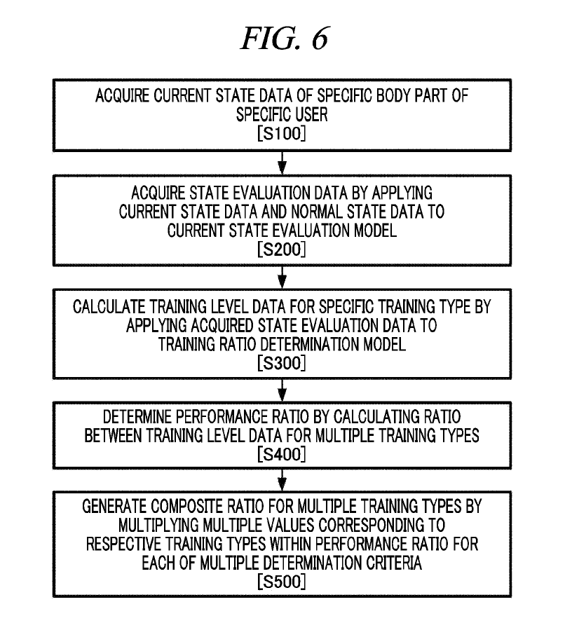

Further, as shown in FIG. 6, in yet another embodiment of the present disclosure, the method may further include: generating a composite ratio for the multiple training types by multiplying multiple values corresponding to the respective training types within the performance ratio for each of the multiple determination criteria (S500). That is, the computer calculates performance ratios for multiple training types according to each of the determination criteria and multiples items corresponding to a specific training type in each of the determination criteria to calculate a composite item value. Then, the computer calculates a ratio between composite item values for the respective training types to determine a composite ratio. Thus, it is possible to calculate a training performance ratio that reflects all the multiple determination criteria for determining training of the user, and the user can be provided with an optimum training curriculum applied with all the determination criteria. The above-described method for determining a training ratio according to an embodiment of the present disclosure may be implemented as a program (or application) and stored in a medium to be combined and executed in the computer which is hardware.

The program may include a code that is coded in a computer language, such as C, C++, JAVA, or a machine language, by which a processor (CPU) of the computer may be read through a device interface of the computer, to execute the methods implemented by a program after the computer reads the program. The code may include a functional code related to a function that defines necessary functions to execute the methods, and the functions may include an execution procedure related control code required to execute the functions by the processor of the computer according to a predetermined procedure. Further, the code may further include additional information required to execute the functions by the processor of the computer or a memory reference related code on a location (address) in an internal or external memory of the computer to be referenced by the media. Further, when the processor of the computer needs to perform communication with another computer or server in a remote site to allow the processor of the computer to execute the functions, the code may further include a communication related code on how the processor of the computer executes communication with another computer or server in a remote site or which information or medium should be transmitted and received during communication by using a communication module of the computer.

The storage medium is a medium that semi-permanently stores data and from which data is readable by a device, but not a medium, such as register, a cache, a memory, or the like, that stores data for a short time. Specifically, examples of the storage medium may include, for example, but not limited to, a read only memory (ROM), a random access memory (RAM), a compact disc (CD)-ROM, a magnetic tape, a floppy disc, an optical data storage device, and the like. That is, the program may be stored in various storage media on various servers which the computer can access or in various storage media on the computer of the user. In addition, the storage medium may be distributed to a computer system connected to a network and a computer-readable code may be stored on a distributed basis in the storage medium.

Although the exemplary embodiments of the present disclosure have been described with reference to the accompanying drawings, it would be understood by a person with ordinary skill in the art that various changes and modifications may be made without changing technical conception and essential features of the present disclosure. Thus, it should be understood that the above-described embodiments are illustrative in all aspects and do not limit the present disclosure.

1. A method for determining a performance ratio of multiple training types of a specific user, comprising:
acquiring current state data for a specific body part of a specific user;
acquiring state evaluation data by applying the current state data and normal state data to a current state evaluation model;
calculating training level data for a specific training type by applying the acquired state evaluation data to a training ratio determination model; and
determining a performance ratio by calculating a ratio of training level data for multiple training types,
wherein the current state data is data for performance of the specific training type for the specific body part of the user,
the normal state data is data for performance of the specific training type for the specific body part of a normal person, and
the state evaluation data is data that is evaluated by comparing the current state data with the normal state data to determine a training level suitable for the current state of the user.

2. The method for determining a training ratio of claim 1,
wherein the current state evaluation model calculates the state evaluation data by calculating a third value corresponding to the state of the user according to a specific calculation equation in a numerical range between a first numerical value corresponding to a minimum state and a second numerical value corresponding to a normal person, and
the calculation equation corresponds to features of the specific body part.

3. The method for determining a training ratio of claim 1,
wherein the training ratio determination model sets the training level data to 0 in the minimum state and a normal state and has an equation which has a specific training level data value in a specific state between the minimum state and the normal state and is set differently depending on the body part or the training type.

4. The method for determining a training ratio of claim 1,
wherein if the body part is a hand, the training type includes rotating a wrist, bending and stretching a wrist, rotating a forearm, and folding and unfolding a finger, and
the current state data is measurement data of a range of motion of a joint for the training type, and
the determining of the performance ratio includes calculating the performance ratio of the multiple training types.

5. The method for determining a training ratio of claim 1,
wherein if the specific training type has a motion that is symmetrical to a reference posture, the state evaluation data of the specific training type is calculated by averaging first state evaluation data corresponding to a motion in a first direction and second state evaluation data corresponding to a motion in a second direction.

6. The method for determining a training ratio of claim 1,
wherein the normal state data is a maximum point on a graph of the number of normal persons for performance result data of the specific training type or an average value of the performance result data acquired from multiple normal persons.

7. The method for determining a training ratio of claim 1,
wherein the current state evaluation model applies the square of a real number greater than 1 to a difference value between the normal state data and the current state data.

8. The method for determining a training ratio of claim 1,
wherein the determining of the performance ratio includes:
acquiring a reference ratio of the multiple training types; and
calculating the performance ratio by multiplying a value corresponding to the specific training type within the reference ratio and the training level data of the specific training type.

9. The method for determining a training ratio of claim 1,
wherein if there are multiple determination criteria for calculating the performance ratio for each training type, the performance ratio for each of the multiple determination criteria is calculated.

10. The method for determining a training ratio of claim 9, further comprising:
generating a composite ratio for the multiple training types by multiplying multiple values corresponding to the respective training types within the performance ratio for each of the multiple determination criteria.

11. A program for determining a training ratio that is combined with a computer which is hardware and stored in a medium to perform a method of claim 1.
